(19) United States
(12) Patent Application Publication
(10) Pub. No.: US 2013/0023859 A1
(43) Pub. Date: Jan. 24, 2013

(54) Articulating Links with Middle Link Control System

(71) Applicant: Jaroslaw T. Malkowski, Trumbull, CT (US)

(72) Inventor: Jaroslaw T. Malkowski, Trumbull, CT (US)

(73) Assignee: Tyco Healthcare Group LP, Mansfield, MA (US)

(21) Appl. No.: 13/546,028

(22) Filed: Jul. 11, 2012

Related U.S. Application Data
(60) Provisional application No. 61/510,091, filed on Jul. 21, 2011.

(51) Int. Cl. A61B 17/00

(52) U.S. Cl. 606/1

(57) ABSTRACT

A surgical instrument that is transitionable between a bent and a straight position is disclosed. The surgical instrument includes two sections that are made up of articulating links that define gaps therebetween to facilitate bending of the surgical instrument. Restricting movement of a middle link that is positioned between the two sections results in a reduced positioned error.

### CROSS-REFERENCE TO RELATED APPLICATIONS

This application claims the benefit of and priority to U.S. Provisional Patent Application No. 61/510,091, filed Jul. 21, 2011, the entire disclosure of which is incorporated by reference herein.

### BACKGROUND

1. Technical Field

The present disclosure relates generally to a surgical instrument including articulating links. More particularly, the present disclosure relates to a surgical instrument including a linkage having a reduced positioning error.

2. Background of Related Art

A minimally invasive surgical procedure is one in which a surgeon enters a patient's body through one or more small openings in the patient's skin or naturally occurring openings (e.g., mouth, anus, or vagina). As compared with traditional open surgeries, minimally invasive surgical procedures have several advantages and disadvantages. Minimally invasive surgeries include arthroscopic, endoscopic, laparoscopic, and thoracoscopic surgeries. Advantages of minimally invasive surgical procedures over traditional open surgeries include reduced trauma and recovery time for patients. The disadvantages include the need to insert many instruments through a single opening and a reduced visualization of the surgical site.

It is critical that a surgeon be able to accurately place surgical instruments within the surgical site. Some surgical instruments are configured to articulate. When articulating a surgical instrument, there may be positioning error. In particular, when a surgical instrument includes articulating links, each link may have a positioning error. While the positioning error of each link may be relatively minor, the cumulative effect of all the positioning errors may be significant. Minimizing such positioning error is desirable to facilitate accurate placement of the instruments within the surgical site.

Consequently, a continuing need exists for improved minimally invasive surgical devices.

### SUMMARY

Disclosed herein is a surgical instrument for use during a minimally invasive surgical procedure.

The surgical instrument is transitionable between a straight and a bent position. The surgical instrument may include an end effector for use in a variety of surgical procedures. In an embodiment, the surgical instrument defines a central longitudinally extending lumen for the reception of a surgical instrument therethrough. The surgical instrument may be used during a minimally invasive surgical procedure and may be placed within a seal anchor port accessing an underlying body cavity.

The surgical instrument includes a distal link, a middle link, and a base link. Each of the distal, middle, and base links defines a longitudinal axis. The angle defined between the longitudinal axes of the distal and base links has a value that is twice that of the angle defined between the longitudinal axes of the middle and base links when the surgical instrument is in the bent position.

The surgical system instrument includes a first segment of articulating links and a second segment of articulating links. Positioned between the first and second segments of articulating links is a middle link having a restricted freedom of rotation. The middle link is positioned between a substantially equal number of articulating links contained in each of the first and second segments. By restricting the freedom of rotation of the middle link with respect to the first and second segments, the positioning error of the distal end of the surgical instrument is reduced.

In an embodiment, a pulley system including cables controls the position of the middle link. In particular, generally opposing cables are looped around pulleys that are secured to or operatively coupled to the middle link. In addition, generally opposing cables are operatively coupled to the distal link. By applying a force to the cables, the surgical instrument is bendable to a desired contour or shape. The surgical instrument may be biased toward the straight position such that when the force ceases to be applied to the cables, the surgical instrument returns to the straight position.

The articulating links contact each other and pivot with respect to each other at contact points. In the straight position, gaps are defined adjacent to the contact points, thereby facilitating bending of the surgical instrument. By restricting movement of the middle link, the size of the gaps between adjacent links is kept substantially equal along any given axis during bending of the surgical instrument. Springs that operatively connect the articulating links to one another at the contact points may be used to bias the surgical instrument towards the straight position.

These and other features of the current disclosure will be explained in greater detail in the following detailed description of the various embodiments.

### BRIEF DESCRIPTION OF THE DRAWINGS

Various embodiments of the present disclosure are described hereinbelow with reference to the drawings, wherein:

FIG. 1 is a perspective view of an embodiment of a surgical instrument in accordance with the present disclosure and shown in an articulated condition;

FIG. 2 is a perspective view of the surgical instrument of FIG. 1 shown in a non-articulated position;

FIG. 3 is a perspective view of an articulating link;

FIG. 4 is an exploded view of the surgical instrument of FIG. 1;

FIG. 5 is a top view of the surgical instrument of FIG. 1;

FIG. 6 is a side view of the surgical instrument of FIG. 1 shown in the articulated position;

FIG. 7 is a cross-sectional, side view of the surgical instrument of FIG. 1;

FIG. 8 is a cutaway side view of a middle link;

FIG. 9 is a perspective view of an embodiment of a surgical instrument shown in an articulated position;

FIG. 10 is a top view of the surgical instrument of FIG. 9;

FIG. 11 is a cross-sectional view of the surgical instrument of FIG. 9; and

FIG. 12 is a perspective view of a seal anchor member shown relative to tissue.

### DETAILED DESCRIPTION OF THE EMBODIMENTS

Particular embodiments of the present disclosure will be described herein with reference to the accompanying drawings. As shown in the drawings and as described throughout the following descriptions, and as is traditional when referring to relative positioning on an object, the term “proximal” will refer to the end of the apparatus that is closest to the clinician during use, and the term “distal” will refer to the end that is farthest from the clinician during use.

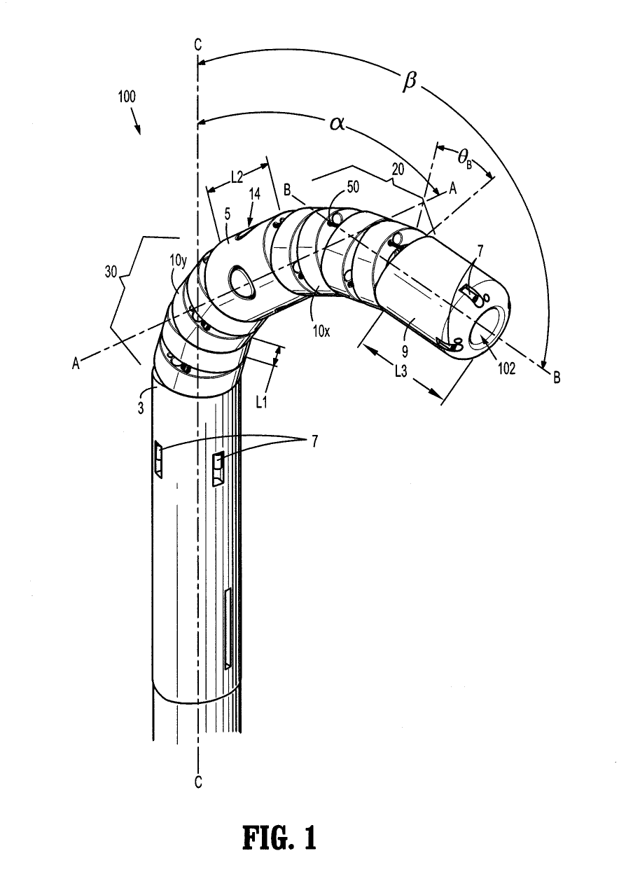

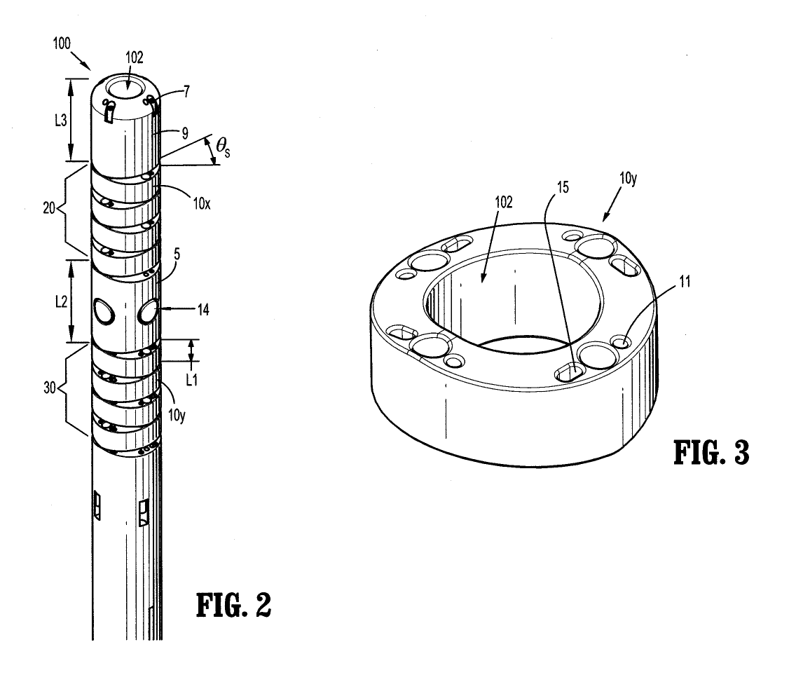

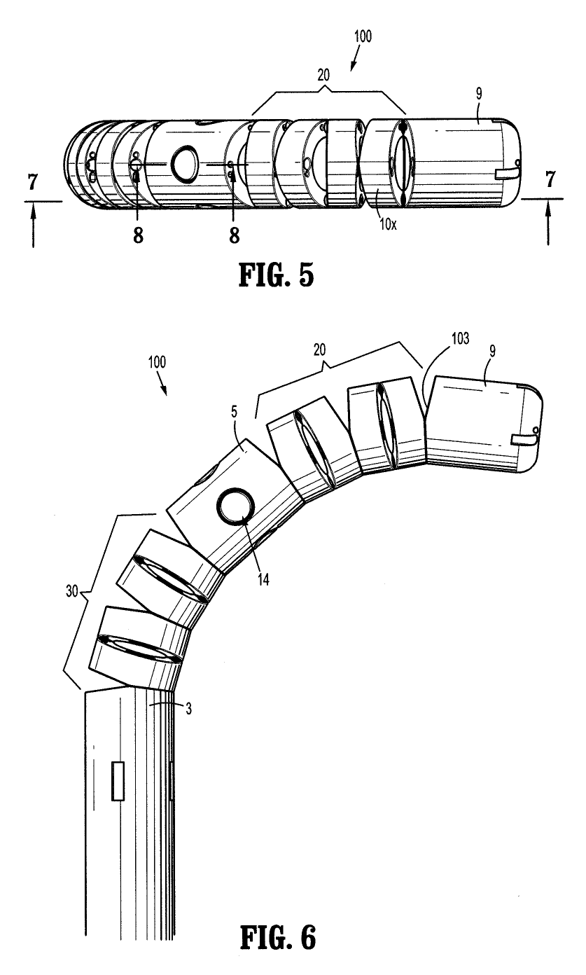

An embodiment of a surgical instrument will now be described with reference to FIGS. 1-8. A surgical instrument 100 is configured and adapted to transition between an articulated or bent condition (FIG. 1) and a non-articulated or straight condition (FIG. 2). The surgical instrument 100 may include a lumen 102 that extends through the surgical instrument 100 and is configured and adapted to receive a surgical instrument therethrough.

The surgical instrument 100 includes at least two segments including adjacent articulating links 10x, 10y. In particular, a first segment 20 includes a plurality of articulating links 10x and a second segment 30 includes a plurality of articulating links 10y. Each segment 20, 30 may include the same number of articulating links. The first segment 20 is positioned distally relative to the second segment 30 and includes a distal link 9. The distal link 9 may be operatively coupled to an end effector (not shown). A middle link 5 is positioned between the first segment 20 and the second segment 30. Preferably, the first segment 20 and the second segment 30 each include a substantially equal number of links 10x, 10y, respectively. A series of cables 50 pass through the second segment 30 and the first segment 20 and are operatively coupled to the distal link 9 to control the positioning of the distal link 9.

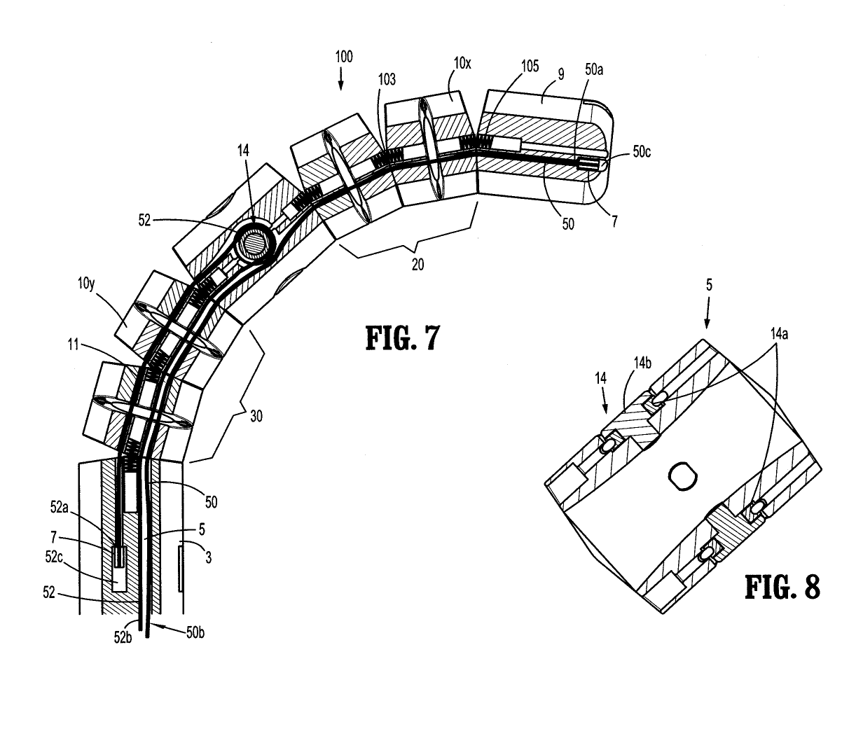

The adjacent links 10x, 10y are shaped and configured to include gaps or spaces between the adjacent links 10x, 10y. The links 10x, 10y contact each other at a contact point 103. The contact point 103 functions as a pivot point for the links 10x, 10y. At the contact points 103, there may be springs 105 (FIG. 7) that bias the surgical instrument 100 toward the unbent or straight position. In particular, each link 10x, 10y may be curved such that the apex of the curve is the contact point 103 between the adjacent links 10x, 10y. The adjacent links 10x, 10y may be arranged such that the contact points 103 are aligned along the same axis such that the gaps between the links 10x, 10y are also aligned co-axially, thereby facilitating bending movement in a single radial direction. In another embodiment, the links 10x, 10y may be arranged and oriented with respect to one another with the contact points 103 alternately aligned, i.e., along a given longitudinal axis, there would be a pattern of a gap followed by a contact point 103, thereby facilitating a bending movement along two radial directions allowing for repositioning to any desired coordinate point. The size of the gap and the value of the angle defined between the gaps is dependent on whether the surgical instrument 100 is bent or straight. As the surgical instrument 100 is bent, the gaps on one side of the surgical instrument will increase while the gaps on the other side of the surgical instrument 100 will decrease.

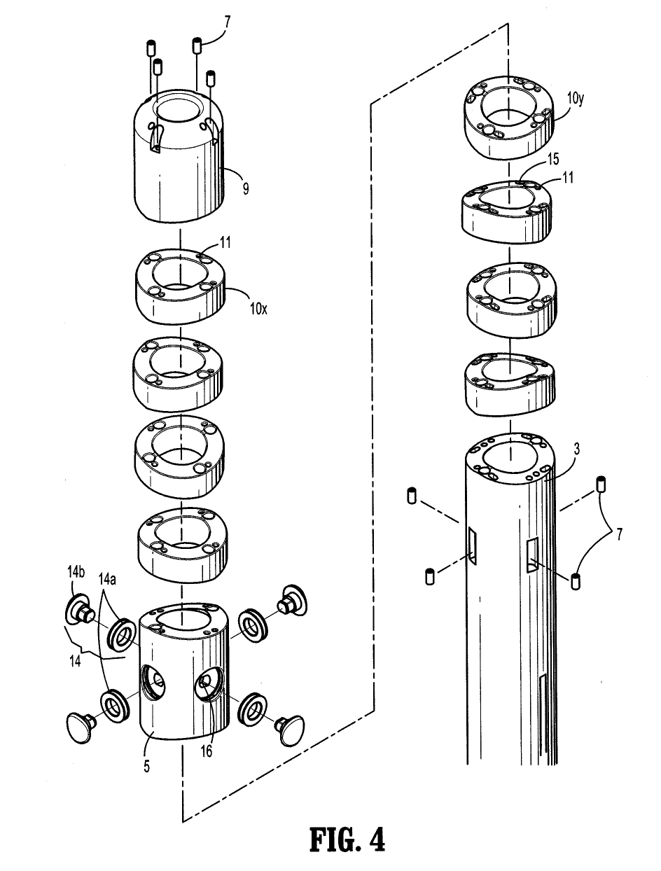

As shown best in FIGS. 3, links 10y of the second segment 30 include a small hold 11 and an elongated hole 15. As shown in FIG. 4, the link 10x includes a small hold 11 for the reception of a single cable, i.e., cable 50. As shown best in FIG. 7, two cables 50, 52 extending from an instrument handle (not shown) pass through each of the elongated holes 15 of links 10y of the surgical instrument 100. Cable 50 only ends through the second segment 30 around a pulley 14a and through small hole 11. The interaction of the cable 52 with the pulley 14a directly controls the position of the middle link 5. In particular, the cable 52 is looped around the pulley 14a which is secured to the middle link 5. The cable 52 is looped around the pulley 14a, effectively doubling the cables acting upon the middle link 5. Application of a force upon the cable 52 in turn applies a force upon the middle link 5, thereby controlling the position of the middle link 5. The middle link 5 may be operatively coupled to several pulleys 14a to facilitate positioning of the middle link 5. For example, the middle link 5 may include four pulleys 14a grouped in generally opposing pairs.

The cable 50 is secured within a distal link 9 adjacent the distal end of the first segment 20. To facilitate radial movement of the distal link 9 in two directions and placement of the distal link 9 at a desired coordinate point, two pairs of opposing cables 50, i.e., four cables 50, may be operatively coupled to the distal link 9. The cable 50 includes a distal end including a ferrule 7 that is secured within a recess 50c defined in the distal link 9. A series of cables 52 only pass through the second segment 30 and are operatively coupled to the middle link 5 to control the positioning of the middle link 5, thereby restricting its freedom of rotation. A first end 52a of the cable 52 is frictionally secured to the base link 3. In particular, the first end 52a of the cable 52 is coupled to a ferrule 7 that is secured within a recess 52s within the base link 3. A second end 52a of the cable 52a and a second end 50a of the cable 50a may extend to a handle (not shown). Application of a force upon the second ends 50a, 52a of cables 50a, 50b, respectively, result in a force being applied to the distal link 9 and the base link 3 respectively.

The middle link 5 may include a plurality of pulley systems 14 that control the actuation of the surgical instrument 100. As shown in FIG. 4 the surgical instrument 100 includes four pulley systems 14. The pulley system 14 includes a pulley 14a around which the cable 52 is looped, and a pin 14b that is frictionally receivable within a recess 16. The pulley systems 14 are positioned in a juxtaposed relationship with one another such that when a force is applied to one of the pulley systems 14, an opposite force can subsequently be applied to a pulley system 14 positioned opposite to bring the second segment 30 surgical instrument 100 back to the original position. Since separate groups of cables 50, 52 are operatively coupled to the distal link 9 and middle link 5, respectively, the first and second segments 20, 30, respectively, are independently actuatable. The cables 50, 52 move together and the pulley system 14 facilitates maintaining a ratio of two to one for the displacement of the distal link 9 relative to the middle link 5. In particular, a force is applied to both the ends 50b, 52b of cables 50, 52, respectively, causing the pair of cables 50, 52 to move together. The positioning of the pulley system 14 with respect to the middle link 5 and the interaction of the cable 52 and the pulley 14a creates the necessary difference in the displacement between the distal link 9 and the middle link 5 to maintain a displacement ratio of 2:1.

By limiting the movement of the middle link 5 relative to the second segment 30, the positioning error of the distal link 9 is minimized. As shown in FIG. 1, the base link 3 defines a longitudinal axis C, the middle link 5 defines a longitudinal axis A, and the distal link 9 defines a longitudinal axis B. The longitudinal axis A of the middle link 5 and the longitudinal axis C of the base link 3 define an angle α therebetween. In addition, the longitudinal axis B of the distal link 9 and the longitudinal axis C of base link 3 define an angle β therebetween. In an embodiment, the middle link 5 is generally evenly centered between the segments 20, 30, and the angle α defined between the middle link 5 and the base link 3 is roughly twice the value as compared to the angle β defined between the distal link 9 and the base link 3 for values of angle β that are between 0° and 96°. In addition, in an embodiment, the angle θₛ between links 10x, 10y when the surgical instrument 100 is straight is approximately 16°. The value of the angle θ_{B} when the surgical instrument 100 is bent is the difference of the angle θ_{S} (angle between links 10x, 10y when straight) multiplied by the number of gaps and the angle β defined between the longitudinal axis B of the distal link 9 and the longitudinal axis C of the base link 3 divided by the number of gaps.

When the surgical instrument 100 is in an extreme position, as in maximally bent, the positioning error is at a minimum. In the maximally bent position, angle β is 96° and angle α is 48°, and the positioning error is zero. When the gaps between the links 10x, 10y are equal, the positioning error is at the theoretical minimum. In particular, when the surgical instrument 100 is bent, the sum of the gaps on one side of the surgical instrument 100 is the sum of the maximum angle β (the angle defined between the longitudinal axis B of the distal link 9 and the longitudinal axis C of the base link 3), i.e., 96°, and the actual angle β (the angle defined between the longitudinal axis B of the distal link 9 and the longitudinal axis C of the base link 3). Each link 10x, 10y has a length that in an embodiment is equal to 0.4000, the middle link 5 has a length L₂ that is equal to 0.8453, and the distal link 9 has a length that is equal to On the other side of the surgical instrument 100, the sum of the gaps is the difference of the maximum angle β, i.e., 96°, and the actual angle β. Where there are 6 gaps between the links 10x, 10y, the Cartesian coordinates, i.e., x and y coordinates, of the theoretical position distal link 9 is given by the following equations: the x-coordinate=L₁*sin(β/6)+L₁*sin((2*β)/6)+L₂*sin((3*β)/6)+L₁*(sin((4*β)/6)+L₁*sin((5*β)/6)+L₃*sin(β) and the y-coordinate=L₁*cos((β/6)+L₁*cos((2*β)/6)+L₂*cos((3*β)/6)+L₁*(cos((4*β)/6)+L₁*sin((5*β)/6)+L₃*cos(β). The actual position of the distal link 9 for values of β that are between 0° and 32°, the x and y coordinates are determined by the following equations: x₁=L₁*sin(16)+L₁*sin(16+β/2)+L₂*sin(β/2)+L₁*sin(β/2+16)+L₁*sin(β/2+16+β/2)+L₃*sin(β) and y₁=L₁*cos(16)+L₁*cos(16+β/2)+L₂*cos(β/2)+L₁*cos(β/2+16)+L₁*cos(β/2+16+β/2)+L₃*cos(β). The actual position of the distal link 9 for values of β that are between 32° and 96°, the x and y coordinates are determined by the following equations: x₂=L₁*sin(16)+L₁*sin(32)+L₂*sin(β/2)+L₁*sin(β/2+16)+L₁*sin(β/2+32)+L₃*sin(β) and y₂=Lₗ*cos(16)+L₁*cos(32)+L₂*cos(β/2)+L₁*cos(β/2+16)+L₁*cos(β/2+32)+L₃*cos(β). The positioning error is determined calculating the difference between the actual position and the theoretical position, i.e., the absolute value of the square root of the sum of the difference of the theoretical x-coordinate and the actual x-coordinate squared and the difference of the theoretical y-coordinate and the actual y-coordinate squared (i.e., |√((x-x₁)²+((y-yl)²)|). In particular, for β=0°, the positioning error is 0.4453, for β=48°, the positioning error is 0.3253, and for β=96°, the positioning error is 0.0000.

When the middle link 5 is positioned between segments of articulating links that have a roughly even number of equally sized links, the positioning error is less than it would be otherwise. In particular, as discussed above, when the surgical instrument is straight, and the movement of the middle link 5 is constrained, angle α and angle β are zero and the maximum positioning error is 0.4453.

However, if the middle link 5 had an unrestricted freedom of movement and was free to rotate, the gaps between the links 10x, 10y would be cumulated in the first segment 20 and the maximum positioning error would be the sum of all of the positioning errors of each link 10x, 10y, and the maximum positioning error would be 1.3440. This is because the gaps between the links 10x contained in the first segment 20 and the gaps between the links 10y contained in the second segment would not have the same value. In addition, the value of angle α would not be equal to half the value of angle β. However, by constraining the movement of the middle link 5, the positioning error of the distal link 9 is greatly reduced since the position of the middle link 5 is not dependent upon the position of adjacent links 10x, 10y and therefore there will not be a cumulative error effect upon the middle link 5.

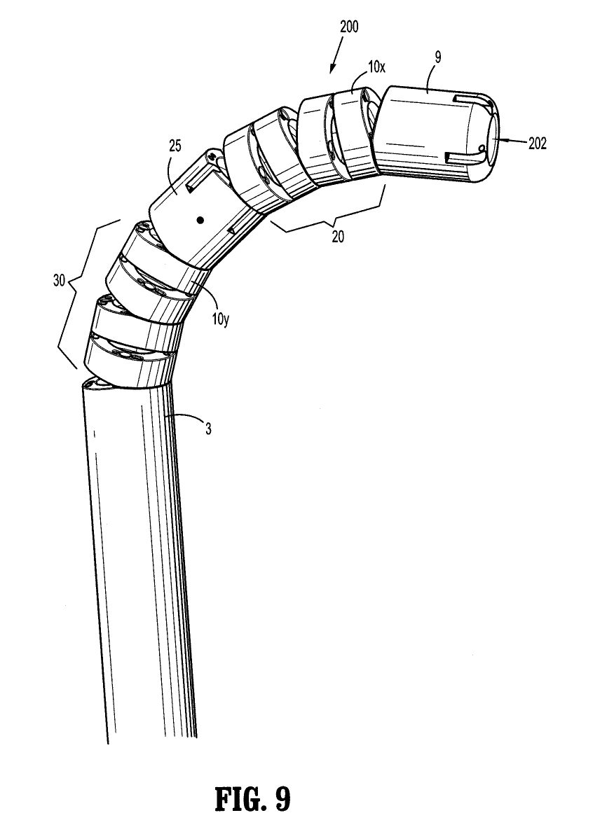

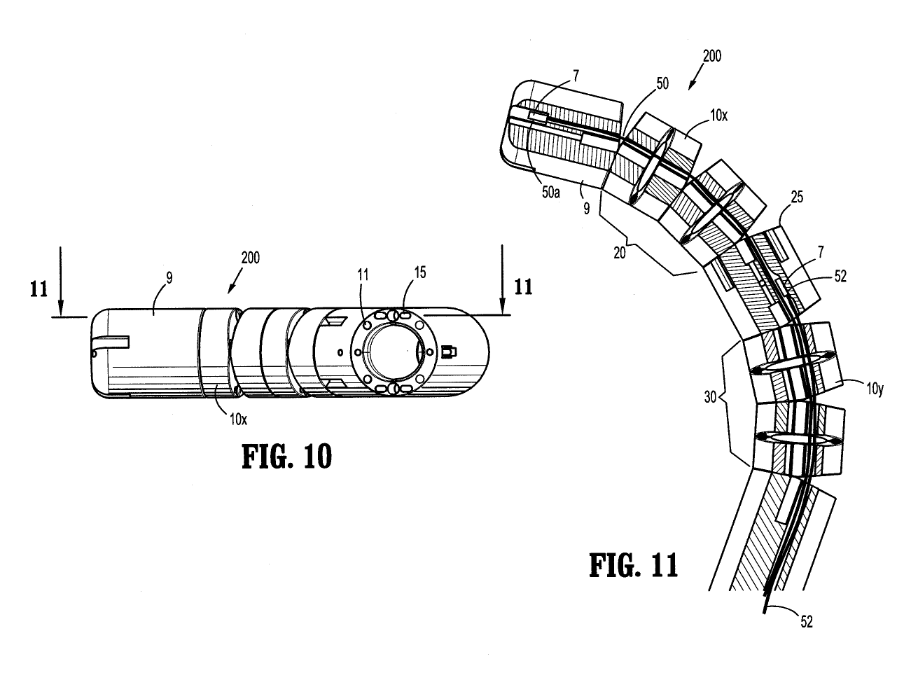

In another embodiment, a surgical instrument 200 does not include a pulley system to effect actuation of the surgical instrument 200. Surgical instrument 200 will now be described with reference to FIGS. 9-11. The surgical instrument 200 is similar to the surgical instrument 100 except that it includes middle link 25 instead of middle link 5. In particular, surgical instrument 200 does not utilize a pulley system. As shown best in FIG. 11, the surgical instrument 200 includes two cables 50, 52 on a lateral side of the surgical instrument 200, e.g., on each of four lateral sides, to facilitate displacement of the surgical instrument to any desired three-dimensional coordinate point. The first cable 50 extends through a first segment 20 of articulating links 10x and a second segment 30 of articulating links 10y and is secured to distal link 9. The second cable 52 extends through the second segment 30 of articulating links 10y and terminates and is secured to the middle link 25. The cables 50, 52 are configured and adapted to displace the distal link 9 relative to the middle link 25 in a ratio of two to one. In particular, the cable 50 that extends to the distal link 9 will move twice as fast as the cable 52 that only extends to the middle link 25 thereby facilitating displacement of the distal link 9 in a two to one ratio relative to the middle link 25.

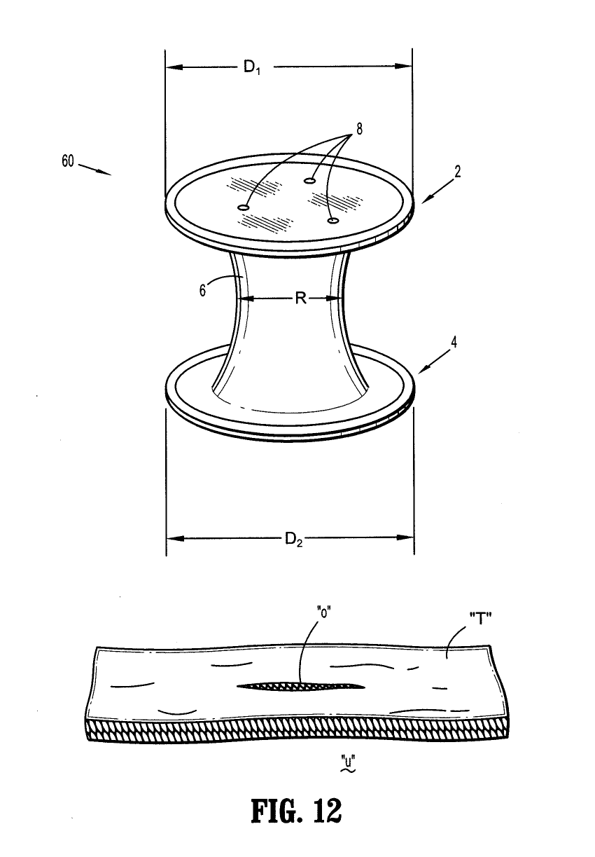

During use a minimally invasive surgery, a surgeon may place a seal anchor member 60 (FIG. 12) within a body opening “O” defined in tissue “T”. The body opening “O” may be naturally occurring (e.g., mouth, anus, vagina) or an incision. The seal anchor member includes a trailing end 2, a leading end 4, and an intermediate section 6. The trailing end 2 defines a diameter D₁, the leading end defines a diameter D₂, and the intermediate section 6 defines a radial dimension that varies along the longitudinal length of the seal anchor member to define a substantially hour-glass shape. The hour-glass configuration of the seal anchor member 60 facilitates the securing of the seal anchor member 60 within the body opening “O” to access an underlying body cavity “U”. Extending longitudinally through the seal anchor member 60 are one or more ports 8 that are configured and adapted for the substantially sealed reception of surgical instruments. An example of a seal anchor member 60 is described in U.S. Pat. Pub. 2009/0093752, the contents of which are hereby incorporated by reference in its entirety.

The surgical instruments 100, 200 are configured and adapted to be placed within the ports 8 of the seal anchor member 60 that is placed within the body opening “O” of tissue “T”. An end effector (not shown) may be operatively coupled to the distal link 9. The end effector chosen is determined based upon the particular application. As discussed above, the positioning of the distal link 9, and the end effector secured thereto, is facilitated by the application of force upon cables 50, 52. The independent actuation of these cables 50, 52 facilitates positioning of the distal link 9 and the end effector.

Although the illustrative embodiments of the present disclosure have been described herein with reference to the accompanying drawings, the above description, disclosure, and figures should not be construed as limiting, but merely as exemplifications of particular embodiments. It is to be understood, therefore, that the disclosure is not limited to those precise embodiments, and that various other changes and modifications may be effected therein by one skilled in the art without departing from the scope or spirit of the disclosure.

What is claimed is:

1. A surgical instrument comprising:
a distal link, the distal link defining a first longitudinal axis;
a middle link, the middle link defining a second longitudinal axis;
a base link, the base link defining a third longitudinal axis;
a first section of articulating links disposed between the distal link and the middle link; and
a second section of articulating links disposed between the middle link and the base link, wherein the second and third longitudinal axes define a first angle, the second and first longitudinal axes define a second angle, the first angle having a value half of the second angle, wherein the surgical instrument is transitionable between a straight position and a bent position.

2. The surgical instrument of claim 1, wherein the articulating links included within each of the first and second sections define gaps therebetween that have substantially equal values along a given axis.

3. The surgical instrument of claim 1, wherein a central longitudinal axis extends through the first and second sections and is configured and adapted to receive an instrument therethrough.

4. The surgical instrument of claim 1, wherein two pairs of generally opposing cables are operatively coupled to the distal link, and wherein another two pairs of generally opposing cables are operatively coupled to the middle link, application of forces upon the cables resulting in bending movement of the surgical instrument.

5. The surgical instrument of claim 5, wherein the middle link includes four pulleys around which the cables that are operatively coupled to the middle link are looped.

6. The surgical instrument of claim 1, wherein the articulating links contact each other at a contact point and pivot with respect to each other at this point.

7. The surgical instrument of claim 6, wherein a spring operatively couples the articulating links to one another at the contact points, thereby biasing the articulating links to an unbent position with respect to one another.

8. The surgical instrument of claim 1, wherein the surgical instrument is biased towards the straight position.

9. The surgical instrument of claim 1, wherein the distal and middle links are independently actuatable.
